(19) United States
(12) Patent Application Publication
(10) Pub. No.: US 2010/0069644 A1
(43) Pub. Date: Mar. 18, 2010

(54) Method of regio-selective synthesis of tri-substituted-1, 2, 3-triazoles

(76) Inventor: Xiaodong Shi, Morgantown, WV (US)

Correspondence Address: WEST VIRGINIA UNIVERSITY RESEARCH CORPORATION, 886 CHESTNUT RIDGE ROAD, P.O. BOX 6224, MORGANTOWN, WV, 26506-6224, US

(21) Appl. No.: 12/584,801

(22) Filed: Sep. 11, 2009

Related U.S. Application Data
(60) Provisional application No. 61/192,113, filed on Sep. 15, 2008.

(51) Int. Cl. C07D 249/04; C07D 403/10

(52) U.S. Cl. 548/255

(57) ABSTRACT

The embodiments provide for region-selective synthesis of tri-substituted 1,2,3-traizoles. A first embodiment provides for the selective N-2 alkylation of a 4,5-disubstituted 1,2,3-triazole. A second embodiment provides for the selective acetylation of a 4,5-disubstituted 1,2,3-triazole. A third embodiment provides for the selective arylation of a 4,5-disubstituted 1,2,3-triazole.

### CROSS-REFERENCE TO RELATED APPLICATIONS

This application claims priority to provisional patent application numbered 61/192,113 filed on Sep. 15, 2008.

### BRIEF DESCRIPTION OF THE SEVERAL VIEWS OF THE DRAWINGS

### DETAILED DESCRIPTION OF THE INVENTION

The different embodiments allow for region-selective synthesis of tri-substituted 1,2,3-triazoles. The embodiments provide for selective N-2 alkylation, acetylation, and arylation of 4,5-disubstituted-1,2,3-triazoles. After the reaction is completed, the resulting solution can be diluted with acetone or another solvent followed by filtration. The filtrate can be condensed under vacuum and the product can be purified by recrystallization using one or more of various solvents, including, water, all alcohol, CH₂Cl₂, acetone, acetonitrile, THF or flash silica gel chromatography using one or more of different solvents, including Hexane, ethyl acetate mix, DCM, alcohol, THF, and acetonitrile to give the N-substituted-1,2,3-triazole.

A first embodiment is a method for the selective N-2 alkylation of 4,5-disubstituted-1,2,3-triazoles. This embodiment allows for various substituted groups on the C-4 and C-5 position of the product. For the 4,5-disubstituted-1,2,3-triazoles, the substituent reactants, a NH-triazole containing AR, R¹ and X, where AR and R¹ can be any combination of alkyl and aryl groups, aryl and aryl groups, or alkyl and alkyl groups and X can be O, CH₂, CHR, and CR₂ where R is any alkyl or aryl. Further, the alkyl groups can be any primary carbon, any secondary carbon or any tertiaryl carbon and their derivatives. The alkylated groups can also be any electrophilic alkyl compounds, such as alkyl halide, alkyl tosylate and alkyl acetate. All of the substituent reactants can be used for N-2 substitution under basic condition with various solvents. For this application basic conditions means a pH value between about 8 to about 14. One or more bases can be used to create basic conditions and the base can be an inorganic base such as NaOH, KOH, and Ca(OH)₂, Cs₂CO₃, K₂CO₃, NaH or an organic base such as Et₃N, NH₃, and pyridine or any other that one skilled in the art would use. The second or R′X reactant can be added to the reaction in one or more solvents. The solvents can be protic such as MeOH, i-PrOH, EtOH, and H₂O or aprotic solvents such as CH₂Cl₂, DMSO, DMF, acetone, acetonitrile, and THF. The R' can be any alkyl while X can be O, CH₂, CHR, and CR₂ where R is any alkyl or aryl. The reaction can be conducted by about 1.0 equivalent of substituent reactant, about 1.0 to about 1.5 equivalents of base, and about 1.0 to about 2.0 equivalents of R′X. Additionally, R′X may be introduced in about 0.1 M to about 0.5 M solvent. The reaction mixture can be stirred at a reaction temperature heated from about 35° C. to about 85° C. or reacted or alternatively at room temperature up to reflux, according to different solvent used and different conditions and different substrates. The reaction continues for a reaction time of about 1 hr to about 72 hr according to different conditions and different substrates. The first embodiment is summarized as below:

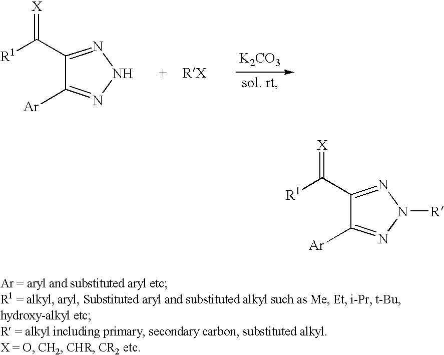

Another embodiment can be used for the selective N-2 acetylation of 4,5-disubstituted-1,2,3-triazoles to allow for various substituted group on the C-4 and C-5 position. This embodiment is summarized as:

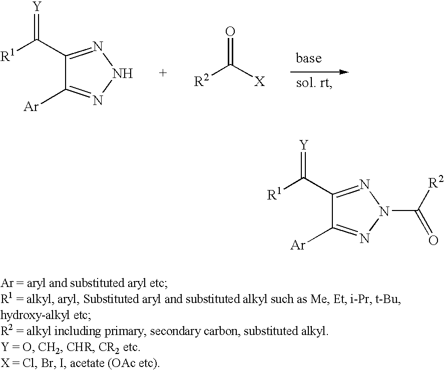

In this embodiment the substituent reactants include an NH-triazole containing R¹, Ar, and Y where R¹ and Ar can be any combination of alkyl and aryl groups, aryl and aryl groups, or alkyl and alkyl groups Y can be O, CH₂, CHR, and CR₂ where R is an alkyl or aryl. The alkyl group can be any primary carbon, any secondary carbon, or any tertiary carbon and their derivatives. The acetylated groups can be any acetyl group including any acyl halide, anhydride, or acetate. The base can be an inorganic base such as Cs₂CO₃, K₂CO₃, NaOH, NaOAc or an organic base such as Et₃N, NH₃, and pyridine or any other that one skilled in the art would use. The acetylated reagent can contain an R² and X where R² is any alkyl and X is Cl, Br, I, or acetate. Additionally, the acetylated reagent may be introduced in solvent. The solvent can be an aprotic or protic solvent which is the same as the alkylation reaction. In addition, some catalysts can be used, such as DMAP, or other Lewis base catalysts. The reaction can be conducted by using about 1.0 equivalent of a substituent reactant, about 1.0 to about 6.0 equivalents of base, about 1.0 to about 5.0 equivalents of the acetylated reagent and, if desired, about 0.01 to about 0.50 equivalents of catalyst. The acetylated reagent may be introduced in about 0.1 to about 0.5 M solvent in solvent. The reaction mixture can be stirred at a reaction temperature range of about 0° C. to about 45° C., or alternatively stirred at a range of room temperature up to reflux, according to different solvent used and different conditions and different substrates. The reaction continues for a reaction time of about 1 hr to about 48 hr according to different conditions and different substrates.

Another embodiment can be used for the selective N-2 arylation of 4,5-disubstituted-1,2,3-triazoles. This embodiment is summarized as:

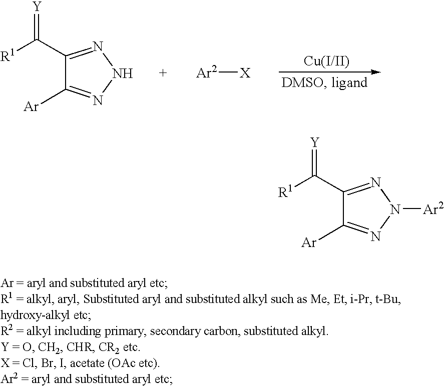

For the selective N-2 arylation of 4,5-disubstituted-1,2,3-triazoles the substituent reactants include an NH-triazole containing Ar, R', and Y where Ar and R¹ can be any alkyl and aryl groups, any aryl and aryl groups, or any alkyl and alkyl groups and Y can be O, CH₂, CHR, and CR₂ where R is an alkyl or aryl. The alkyl group can be any primary carbon, secondary carbon or tertiary carbon and their derivatives. The arylated groups can be any aryl group including aryl halide, aryl tosylate and aryl acetate, aryl boric acid or boronate esters. The substituted reactants for the arylated groups can be electron-donating or electron-withdrawing group. In order to perform this embodiment a mixture of about 1.0 equivalent of substituent reactant, an effective amount of a solvent such as about 0.2 M DMSO, about 1.0 to about 2.5 equivalents of Ar²—X where X═Cl, Br, or I and Ar² is the arylated group, may be added to a catalytic amount which ranges from 1% to 100% of cupper salt which can be any copper I salts or copper II salts, such as CuI, CuCl, Cu(OAc)₂, CuSO₄, Cu(acac)₂ and a catalytic amount, from 1% to 100%, of ligands which include the ligands such as proline, bi-pyridine, glycine, N-methylglycine, N,N-dimethylglycine, cyclohexyldiamene, dmeda, TMEDA. The resulting reaction mixture can be stirred at a reaction temperature ranging from room temperature to 180° C. and monitored by TLC.

The different embodiments of alkylation, acetylylation, and arylation reactions using different electrophilic alkyl, acetyl, and aryl substituents with region-selective substitutions can provide for many different products. Some examples are:

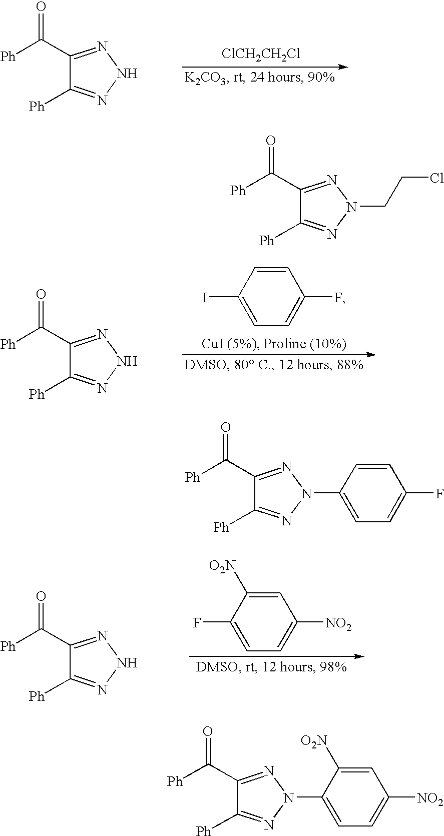

The embodiments can be used to produce any of the following types of products among many other substituents that can be added.

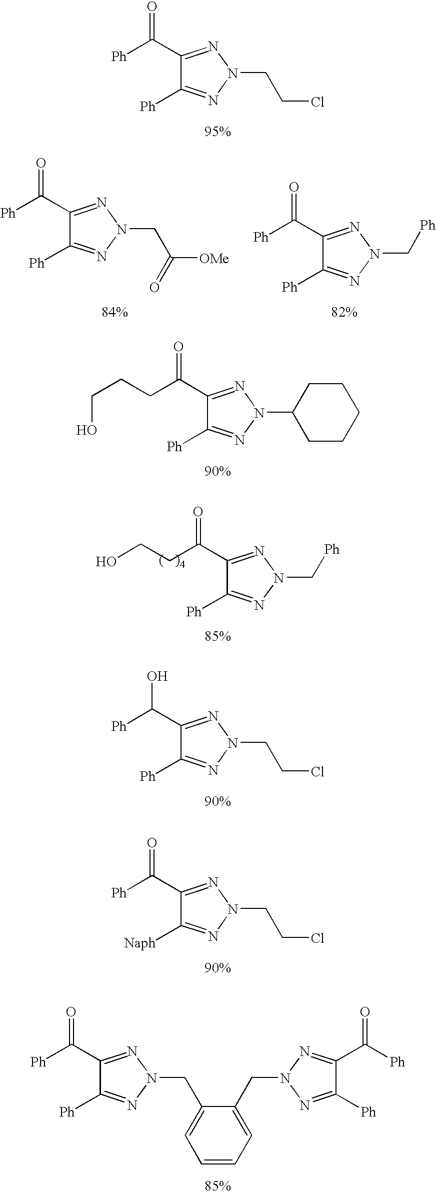

These terms and specifications, including the examples, serve to describe the invention by example and not to limit the invention. It is expected that others will perceive differences, which, while differing from the forgoing, do not depart from the scope of the invention herein described and claimed. In particular, any of the function elements described herein may be replaced by any other known element having an equivalent function.

What is claimed is:

1. A method comprising a selective N-2 alkylation of 4,5-disubstituted-1,2,3-triazoles reaction wherein about 1.0 equivalent of 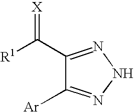
wherein Ar is an alkyl or aryl group, R¹ is an alkyl or aryl group, and X is one of O, CH2, CHR and CR₂ wherein R is an alkyl or aryl group is added about 1.0 to about 1.5 equivalents of base to about 1.0 to 2.0 equivalents of R′X where R' is any alkyl and X is one of O, CH2, CHR and CR₂ wherein R is an alkyl or aryl at a reaction temperature for a reaction time to yield 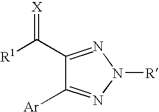
wherein R¹ is an alkyl or aryl group, X is one of O, CH2, CHR and CR₂ wherein R is an alkyl or aryl group and R′ is any alkyl.

2. The method of claim 1 wherein said alkyl is one or more of a primary carbon, a secondary carbon, a tertiary carbon, or a derivative thereof.

3. The method of claim 1 further comprising dissolving said R′X in about 0.1 M to about 0.5 M solvent before R′X addition.

4. The method of claim 3 wherein said solvent is one or more of MeOH, i-PrOH, EtOH, H₂O, CH₂Cl₂, DMSO, DMF, acetone, acetonitrile, and THF.

5. The method of claim 1 wherein said base is one or more of NaOH, KOH, Ca(OH)₂, Cs₂CO₃, K₂CO₃, NaH, Et₃N, NH₃, and pyridine as needed to create basic conditions for said reaction.

6. The method of claim 1 wherein said aryl is substituted.

7. A method comprising a reaction for the selective N-2 aceelyation of 4,5-disubstituted-1,23-triazoles where about 1.0 equivalents of 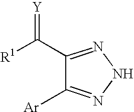
wherein Ar is an alkyl or aryl group, R¹ is an alkyl or aryl group, and Y is one of O, CH2, CHR and CR₂ and R is an alkyl or aryl group is added to about 1.0 to about 6.0 equivalents of base, about 1.0 to about 5.0 equivalents of acetylated reagent 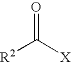
wherein R² is an alkyl and X is Cl, Br, I, or acetate at a reaction temperature for a reaction time to yield 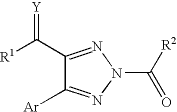
wherein Ar is an alkyl or aryl group, R¹ is an alkyl or aryl group, and Y is one of O, CH2, CHR and CR₂, R is an alkyl or aryl group and R² is an alkyl and X is Cl, Br, I, or acetate.

8. The method of claim 7 wherein said alkyl is one or more of a primary carbon, a secondary carbon, a tertiary carbon, or a derivative thereof.

9. The method of claim 7 wherein said alkyl is an electrophilic alkyl.

10. The method of claim 7 wherein said base is one or more of Cs₂CO₃, K₂CO₃, NaOH, NaOAc, Et₃N, NH₃, and pyridine.

11. The method of claim 7 wherein 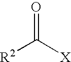
can be introduced in about 0.1M to about 0.5M solvent.

12. The method of claim 7 wherein said solvent is one or more of MeOH, i-PrOH, EtOH, H₂O, CH₂Cl₂, DMSO, DMF, acetone, acetonitrile, and THF.

13. The method of claim 7 further comprising the addition of about 0.1 to about 0.50 equivalents of catalyst wherein said catalyst is a Lewis base catalyst.

14. The method of claim 13 wherein said catalyst is DMAP.

15. The method of claim 7 wherein said aryl is substituted.

16. A method comprising a reaction for the selective N-2 arylation of 4,5-disubstituted-1,2,3-triazoles where about 1.0 equivalent of 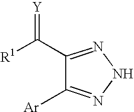
wherein Ar is an alkyl or aryl group, R¹ is an alkyl or aryl group, and Y is one of O, CH2, CHR and CR₂, R is an alkyl or aryl group is added to an effective amount of a solvent, about 1.0 to about 2.5 equivalents of Ar²—X wherein Ar² is an aryl and X is Cl, Br, or I and a catalytic amount of copper salt is added, and a catalytic amount of ligands are added at a reaction temperature to yield 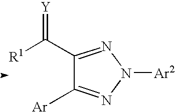
wherein Ar is an alkyl or aryl group, R¹ is an alkyl or aryl group, Y is one of O, CH2, CHR and CR₂, R is an alkyl or aryl group and Ar² is an aryl.

17. The method of claim 16 wherein said solvents is 0.2 M DMSO.

18. The method of claim 16 wherein said catalytic amount of copper salt is about 1% to about 100% of CuI, CuCl, Cu(OAc)₂, CuSO₄, or Cu(acac)₂.

19. The method of claim 16 wherein said catalytic amount of ligands is about 1% to about 100% of proline, bi-pyridine, glycine, N-methylglycine, N,N-dimethylglycine, cyclohexyldiamene, dmeda, TMEDA.

20. The method of claim 16 wherein said aryl is substituted.
